(19) United States
(12) Patent Application Publication
(10) Pub. No.: US 2024/0115181 A1
(43) Pub. Date: Apr. 11, 2024

(54) SQUEEZE AND STICK SAFETY SUBDERMAL NEEDLE ELECTRODE

(71) Applicant: Rhythmlink International, LLC, Columbia, SC (US)

(72) Inventor: Brian PETREE, West Columbia, SC (US)

(21) Appl. No.: 18/484,932

(22) Filed: Oct. 11, 2023

Related U.S. Application Data
(60) Provisional application No. 63/415,035, filed on Oct. 11, 2022.

(51) Int. Cl. A61B 5/262; A61B 5/263; A61B 5/291; A61B 17/34

(52) U.S. Cl. CPC A61B 5/262; A61B 5/263; A61B 5/291; A61B 17/3403

(57) ABSTRACT

Disclosed are various aspects for a squeeze and stick subdermal needle electrode. A squeeze and stick subdermal needle electrode may comprise (a) an electrode housing with (i) an inner portion with an opening to receive a subdermal needle on a proximal side, and a lead wire on a distal side, wherein the subdermal needle and the lead wire are mated together to transfer electrical signals; and (ii) an outer portion for protecting the inner portion, the outer portion capable of compressing onto the inner portion, wherein when compressing drives the subdermal needle toward a patient. Further, methods of compressing the outer portion to drive the inner portion of the subdermal needle electrode into a subdermal region of the patient are disclosed.

### CROSS-REFERENCE TO RELATED APPLICATIONS

The present application is related to and claims the benefit and priority of U.S. Provisional Patent Application No. 63/415,035, filed on Oct. 11, 2022, entitled Squeeze and Stick Safety Subdermal Needle Electrode, the contents of which are incorporated herein.

### FIELD

The present invention relates to neurological monitoring, in particular to systems and methods relating to subdermal needle electrodes.

### BACKGROUND

Subdermal needle electrodes are utilized in intraoperative neuromonitoring (IONM) and during an electroencephalogram (EEG) to monitor, record, and elicit bio-signals. IONM protects patients by continuously monitoring the central nervous system (brain, spinal cord, and nerves) during medical operations. The real time information from IONM can work to prevent neurological injury, and further, allow a medical professional to respond with accuracy to minimize long-term post-operative damage. Neurological monitoring allows practitioners to develop insight into the brain and neurological system.

Subdermal needle electrodes, when used for EEG, have the ability to insert into a subdermal region of the skin, and eliminate the need to abrade the skin to achieve low impedance. Therefore, reducing the amount of time needed to apply multiple electrodes, and further improving patient care.

Subdermal needle electrodes may be applied to specific locations through templates and the use of markers on the skin to indicate positioning. Often times there are a plurality of subdermal needle electrodes applied, each with a lead, and returning to a monitor that displays the electrical signals.

In situations of applying a high numerosity of subdermal needle electrodes, extra care is required so as to correctly position the needle electrodes, while also not causing improper needle sticks, or inaccurate needle sticks to the patient or the user. In the hospital environment this is often a time sensitive matter, one in which users must have some degree of skill and dexterity. Needle stick injuries can be quite costly, and risk the transmission of disease.

Thus, there is a long sought need to improve patient care and user experience by providing rapid deployment of a subdermal needle electrode, wherein the needle is comprised of low cost materials, and is designed to be guarded to a patient and a user, and may be applied quickly by a mechanism that is reliable and safe. In turn reducing improper needle sticks and improving clinical care. Therefore, the disclosure herein provides for a squeeze and stick safety subdermal needle electrode along with related systems and methods.

### SUMMARY

In some aspects, the techniques described herein relate to a squeeze and stick subdermal needle electrode, including: (a) an electrode housing, including: (i) an inner portion including a semi-rigid projection with a top surface sloped downward from a medial line, the semi-rigid projection with an opening to receive a subdermal needle on a proximal side, and a lead wire on a distal side, wherein the subdermal needle and the lead wire are mated together to transfer electrical signals; and (ii) an outer portion including a left side and a right side of semi-rigid material for protecting the inner portion, the outer portion capable of compressing into the inner portion, wherein the compressing drives the left side and the right side of the outer portion into the top surface of the semi-rigid projection which drives the subdermal needle toward a patient.

In some aspects, the techniques described herein relate to a subdermal needle electrode, wherein the electrode housing is included of a polymeric material.

In some aspects, the techniques described herein relate to a subdermal needle electrode, wherein the inner portion is included of a polymeric material and the outer portion is included of a metal.

In some aspects, the techniques described herein relate to a subdermal needle electrode, wherein the subdermal needle is included of stainless steel and a non-tip portion of the subdermal needle has a protective coating.

In some aspects, the techniques described herein relate to a subdermal needle electrode, further including the outer portion having an angular left side and right side that when acted upon compresses the outer portion into the inner portion at a respective angle that causes the outer portion to slide above the inner portion to drive the subdermal needle toward the patient.

In some aspects, the techniques described herein relate to a subdermal needle electrode, further including the outer portion having the left side and the right side, wherein when the left side and the right side are connected to form a circle that provides protection from the subdermal needle

In some aspects, the techniques described herein relate to a subdermal needle electrode, wherein the proximal side of the inner portion is configured with an adhesive to secure and support the subdermal needle.

In some aspects, the techniques described herein relate to a subdermal needle electrode, wherein the subdermal needle and the lead wire are mated with a solder alloy.

In some aspects, the techniques described herein relate to a subdermal needle electrode, further including an insulation wrap, wherein the insulation wrap is wrapped around the solder alloy, the subdermal needle, and the lead wire.

In some aspects, the techniques described herein relate to a subdermal needle electrode, wherein the outer portion is open on one end to allow room for the subdermal needle to depress into the patient.

In some aspects, the techniques described herein relate to a method for deploying a squeeze and stick needle electrode, including: provisioning a squeeze and stick subdermal needle electrode, including: (a) an electrode housing, including: (i) an inner portion including a semi-rigid projection with a top surface sloped downward from a medial line, the semi-rigid projection with an opening to receive a subdermal needle on a proximal side, and a lead wire on a distal side, wherein the subdermal needle and the lead wire are mated together to transfer electrical signals; and (ii) an outer portion including a left side and a right side of semi-rigid material for protecting the inner portion, the outer portion capable of compressing into the inner portion; compressing the left side and the right side of the outer portion into the top surface of the semi-rigid projection, which drives the subdermal needle toward a patient.

In some aspects, the techniques described herein relate to a method, further including releasing compressing forces from the left side and the right side of the outer portion once the subdermal needle has entered the patient.

In some aspects, the techniques described herein relate to a method, further including removing the subdermal needle electrode by moving in an opposite direction from insertion, wherein the removing of the subdermal needle allows the subdermal needle to retract back into the outer portion guarding the subdermal needle from improper needle sticks.

In some aspects, the techniques described herein relate to a method, wherein the electrode housing is included of polymeric material.

In some aspects, the techniques described herein relate to a method, wherein the inner portion is included of a polymeric material and the outer portion is included of a metal.

In some aspects, the techniques described herein relate to a method, wherein the subdermal needle is included of stainless steel and a non-tip portion of the subdermal needle has a protective coating.

In some aspects, the techniques described herein relate to a method, further including the outer portion with an angular left side and right side that when acted upon compresses the outer portion into the inner portion at a respective angle that causes the outer portion to slide above the inner portion to drive the subdermal needle toward the patient.

In some aspects, the techniques described herein relate to a method, further including the outer portion having the left side and the right side, wherein when the left side and the right side are connected to form a circle that provides protection from the subdermal needle

In some aspects, the techniques described herein relate to a method, wherein the outer portion is open on one end to allow room for the subdermal needle to depress into the patient.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. In the drawings:

FIG. 1A is an illustration of an example squeeze and stick subdermal needle electrode in a pre-deployment state;

FIG. 1B is an illustration of an example squeeze and stick subdermal needle electrode in a deployment state;

FIG. 1C is an illustration of an example squeeze and stick subdermal needle electrode in a post-deployment state;

FIG. 2A is an illustration of an example squeeze and stick subdermal needle electrode;

FIG. 2B is an additional illustration of an example squeeze and stick subdermal needle electrode in a pre-deployment state;

FIG. 2C is an additional illustration of an example squeeze and stick subdermal needle electrode in a deployment state;

FIG. 3 is a perspective illustration of an example squeeze and stick subdermal needle electrode;

FIG. 4A is a perspective illustration of an example squeeze and stick subdermal needle electrode;

FIG. 4B is a side illustration of an example squeeze and stick subdermal needle electrode; and

FIG. 5 is a flow chart of an example method of deploying a squeeze and stick subdermal needle electrode.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

In one aspect, a squeeze and stick subdermal needle electrode is utilized to protect clinicians and patients from accidental needle stick. The disclosure herein provides for sheathing or protecting of the subdermal needle while it is not in user or not deployed. Upon compressing and deploying, a movement associated with moving from a pre-deployment state to a deployment state, and ultimately, a post-deployment state. In a deployment state the subdermal needle is in “attack” position for entering the subdermal region of a patient to acquire or transmit electrical signals.

In other aspects, the squeeze and stick subdermal needle electrode provides a one handed design that facilitates usage by clinicians. The configuration of the subdermal needle electrode further provides optimal geometry for inserting the subdermal needle at an appropriate angle to penetrate the subdermal region. Thus, the disclosure herein further facilitates clinicians who must perform the methods of applying and removing a numerosity of subdermal needle electrodes in brief succession.

In further aspects, the polymeric design allows for low cost and rapid manufacture, thus serving economic and environmental benefits. In one aspect the electrode housing may be comprised of a recyclable polymeric material, and may be designed to be disposable within current hospital ecosystems for sharps and other needles. In another aspect, the singular electrode housing eliminates moving parts and further reduces risk of failure by reducing part count and complexity.

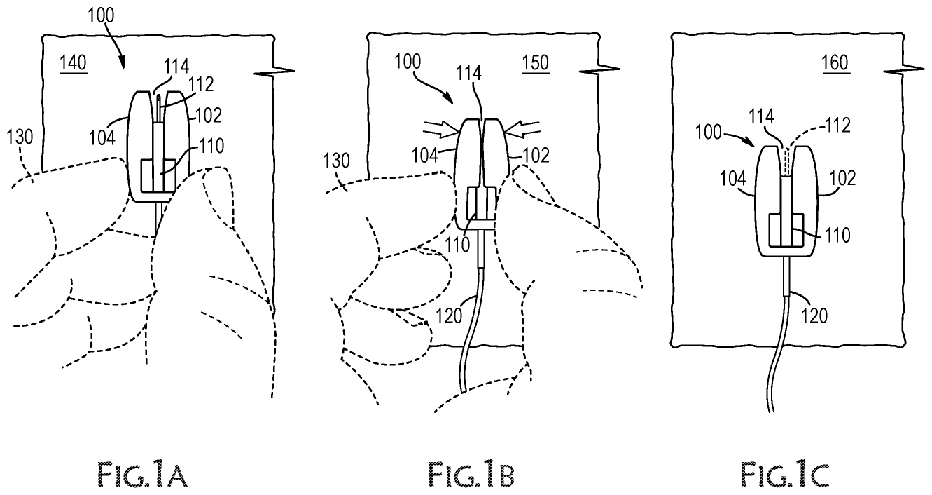

Referring now to the drawings. In FIGS. 1A-C, example aspects of a squeeze and stick subdermal needle are disclosed as it moves through various orientations of a pre-deployment state, deployment state, and a post deployment state, respectively. FIG. 1A discloses an example squeeze and stick subdermal needle electrode in a pre-deployment state 140. In the example, a practitioner 130 is placing the subdermal needle electrode within a relative zone for acquiring electrical signals. In one aspect, this template may be provided so that the practitioner may be guided to the respective areas for placement of the subdermal needle electrode. Further, the subdermal needle electrode is oriented for the insertion site 100 toward the target insertion zone. Continuing, in this aspect, the outer portion has a left side 104 and a right side 102 used for shielding the inner portion 410 comprising a semi-rigid projection and the subdermal needle 112. In this configuration one end is open forming a U shape that allows the subdermal needle 112 to move through the opening when transitioning to a deployment state. In this aspect the outer portion, comprising the left side 104 and the right side 102 fully encompass the subdermal needle 112, and serves as a mechanism for moving the inner portion into an angle for deployment, as well as a sheathing from accidental needle sticks.

Continuing, in FIG. 1B, an example squeeze and stick subdermal needle electrode is disclosed in a deployment state 150. In this aspect the practitioner 130 has applied mechanical force to the left side 104 and right side 102 of the outer portion of the subdermal needle electrode. In doing so the inner portion 110 is driven downward to an angle of 15 to 90 degrees, depending on the patient. The insertion angle will vary based on patient characteristics, but should conform to sending and receiving electrical signals from a subdermal region. In the example a cable 120 is shown running from the inner portion 410 comprising a semi-rigid projection, to the subdermal needle electrode. Furthermore, the subdermal needle is depicted within the subdermal region of the patient and at the insertion zone or insertion site 100.

Continuing, FIG. 1C is an illustration of an example squeeze and stick subdermal needle electrode in a post-deployment state 160. In this aspect, the subdermal needle electrode is fully deployed and the subdermal needle is placed in the subdermal tissue of a patient. The left side 104 and the right side 102 are retracted back so as to no longer place driving force on the inner portion 410, comprising a semi-rigid projection. In this aspect the subdermal needle is in position for sending/receiving electrical signals from a patient and transmitting the electrical signals along a cable 120, and into an instrument for interpretation and further processing. The connection of the subdermal needle and the cable 120 may occur within the inner portion 110, and may be soldered together with a metal alloy, and further wrapped with an insulating wrap. In some aspects, a glue may also be applied to hold the subdermal needle in fixed position within the inner portion 110. In other aspects, only an insulating wrap may be applied, and the subdermal needle may be compression fit or thermally affixed to the inner portion 110.

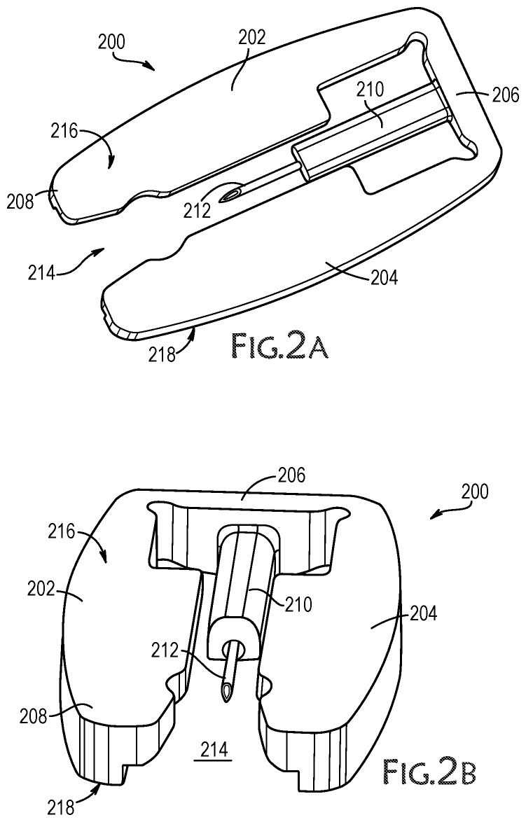

Referring now to FIGS. 2A-C, aspects of a squeeze and stick subdermal needle electrode moving from a pre-deployment state, to a deployment state, and a post-deployment state, respectfully. In the example of FIG. 2A, a subdermal needle electrode is disclosed with an outer portion 206 comprising a left side 204 and a right side 202. The left and right side serve as protection and support for the subdermal needle 212, as well as the driving mechanism for mechanical force onto the inner portion 210. The inner potion 210 comprising the subdermal needle 212 and a cable in which to transmit the electrical signals. Continuing, the outer portion is comprised of a right protective end 216 and a left protective end 218 that protects the subdermal needle from accidental needle stick. The outer portion may further have an opening or a need insertion area 214 wherein the subdermal needle may be retracted downward into position for inserting into subdermal tissue. The outer portion may further be beveled or angled in the opposite degree to the angle on the inner portion 210 to allow for downward force against the semi-rigid inner portion. In some aspects, the outer portion is more rigid than the inner portion, to allow for the outer portion to act upon the inner portion. In other aspects the inner and outer portions may be created from the same semi-rigid material and be sized to allow the respective forces to place pressure on the top surface of the inner portion and to drive the inner portion at an angle for subdermal needle insertion.

Continuing, FIG. 2B discloses another aspect of the subdermal needle electrode of FIG. 2A. In this aspect the inner portion 210 is disclosed depicting the subdermal needle 212 and the open area of the outer portion also known as the insertion area 214. In this aspect the left side 204 and the right side 202 are disclosed with a bevel on the interior surface for deflecting the inner portion 210 toward a patient. The outer portion 206 may be configured to provide flex along a back wall to allow the left side 204 and right side 204 of the outer portion to retract after compressive forces have been applied. In this regard, the material of choice is a semi-rigid polymeric or metal, that holds memory of position, and can flex under mechanical force. The material optimally would be shielded from electrical signals or display little conductance. In the aspect of metal components, they may be wrapped in polymeric material or other insulating material. For example, the outer portion 206 may have an inner skeleton of metal, and an outer layer of polymeric material.

Continuing, in FIG. 2B, the left side 204 and the right side 206 of the outer portion may be a single piece with the inner portion 210, wherein the outer portion 206 is designed to flex into the inner portion 210, to drive the subdermal needle into position for placement in subdermal tissue of a patient. The outer portion 206 may have a right and left protective end 216 that extends past the threshold of the subdermal needle.

Continuing, in FIG. 2C, a deployment state 200 is disclosed with the left side 204 and the right side 202 being mechanically compressed to drive the inner portion 210 to an angle of attack between 15 to 90 degrees for entry into subdermal tissue. The subdermal needle 212 is angled to pierce the patients dermal tissue to enter a subdermal region. The left and right protective ends remain in place and allow the subdermal needle to pass through the insertion area 214.

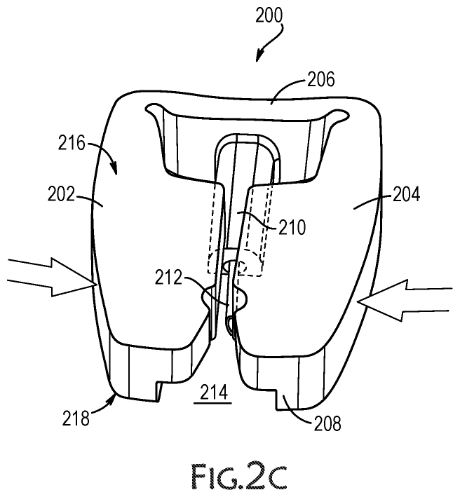

Referring to FIG. 3, an additional example of a squeeze and stick subdermal needle electrode is disclosed. In this aspect the outer portion 306 forms a complete circle or oval, with an indentation or gap 314 for the subdermal needle 312 to remain sheathed in a pre-deployment state. The gap 314 is a region within the outer portion 306 that serves as protection for the subdermal needle, and offers protection for the practitioner and patient from accidental needle stick when handling. The inner portion 310 blends seamlessly with the outer portion 306, and the inner portion 310 provides stability for the subdermal needle, as well as protects the connection between the subdermal needle and the cable assembly.

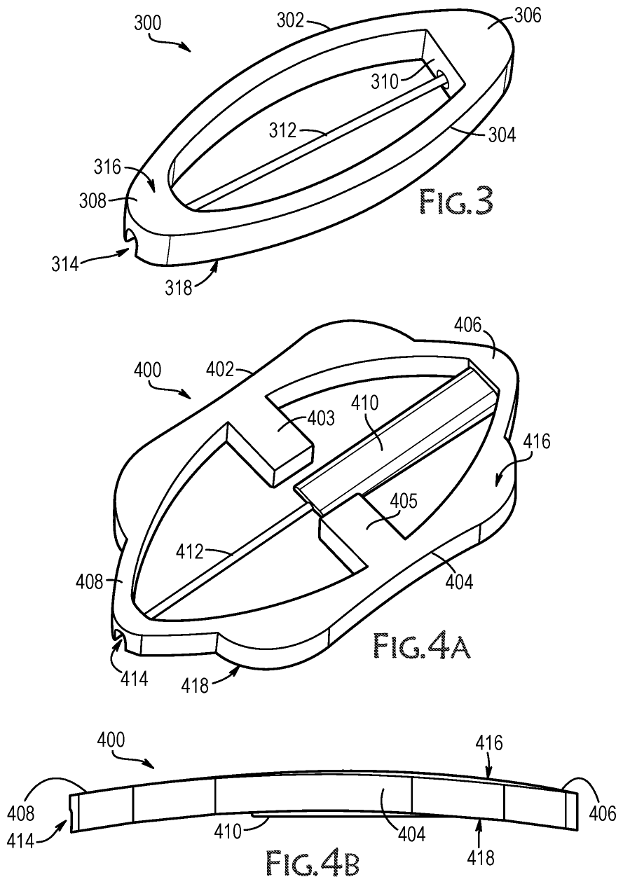

Continuing, with reference to FIGS. 4A-B, in another aspect, a squeeze and stick subdermal needle electrode 400 comprises (a) an electrode housing. The electrode housing comprises (i) an inner portion 410 comprising a semi-rigid projection with an opening to receive a subdermal needle 412 on a proximal side, and a lead wire on a distal side (not shown), wherein the subdermal needle 412 and the lead wire are mated together to transfer electrical signals within the inner portion 410. This mating may be conducted through soldering of a metal alloy, by wrapping and providing insulation, or by other physical connection. The inner portion 410 comprising an angular design, similar to a roof structure, wherein the medial line is the highest point on the top surface of the an inner portion 410 comprising a semi-rigid projection, and the remaining top surface is pitched or sloped to configure with the outer portion 406. The outer portion 406 is assembled for protecting the inner portion 410, and for administering the subdermal needle 412 to a patient by compressive forces. The outer portion 406 comprising a left side 404 and a right side 402 of semi-rigid material, wherein the left side 404 and right side 402 are designed for compressing onto the inner portion 410 comprising a semi-rigid projection, wherein when compressing drives the subdermal needle 412 toward a patient. This compression occurs due to the angles/geometry of the inner portion 410 and the outer portion 406, so that the outer portion 406 moves angularly alongside the inner portion 410, wherein the outer portion 406 having more rigidity and force to drive the inner portion downward at an angle for subdermal needle insertion.

In one aspect, the inner portion 410, comprising a semi-rigid projection, serves as a receptacle for the subdermal needle 412 and the lead wire, and further provides support for the subdermal needle 412 so that it can be applied without bending or breaking. The outer portion 406 of the electrode housing provides for safety and protection of the subdermal needle 412, as well as a semi-rigid structure that the practitioner may hold onto without risk of accidental needle sticks.

In another aspect, the subdermal needle 412 is comprised of stainless steel and a non-tip portion of the subdermal needle 412 has a protective coating. In said aspect the coating may be Teflon™ or other product that may inhibit bacterial growth and/or decomposition of the stainless steel needle.

In another aspect, the inner portion 410 of the subdermal needle electrode housing 400 is comprised of an angular top that when acted upon by the outer portion 406 causes the outer portion 406 to slide above the inner portion to drive the subdermal needle 412 toward the patient. In this aspect, the inner portion 410 is capable of flexing under pressure from the outer portion 406, thus driving the needle to an “attack” or insertion angle for a subdermal placement. In some aspects a front edge guide 414 holds the subdermal needle 412 in place and shortens the overall length of the subdermal needle housing 400 by embedding the subdermal needle 412 into the housing. The subdermal needle electrode housing 400 and the inner portion 410 and outer portion 406 are comprised of a top side 416 and a bottom side 418, wherein the bottom side 418 is placed against the patient.

In another aspect, the subdermal needle electrode housing 400 has an outer portion 406 comprising a left side 404 and a right side 402, wherein when the left side 404 and the right side 406 are compressed when moving from a pre-deployment state to a deployment state, and act upon the inner portion 410 to drive the subdermal needle 412 toward the patient. In this aspect, the polymeric material and layer thickness of the outer portion are enough to maintain integrity of the apparatus, but allow some degree of compressibility in which to compress on and drive the inner portion comprising the subdermal needle at an angle for insertion into a patient. Thus, the outer portion 406 provides protection for both the clinician and the patient while allowing the subdermal needle 412 to be readily deployed via compression of the outer layer 406.

In another aspect, the proximal side of the inner portion 406 of the subdermal needle electrode housing is configured with an adhesive to secure and support the subdermal needle 412. In other aspects, the adhesive is also used to connect the lead wire, and may further be wrapped in an insulating wrap. In other aspects, the subdermal needle 412 is manufactured or melted into the inner portion 406 of the subdermal needle electrode housing 400, resulting in an integrated unit. In further aspects, a proximal end on the inner portion 410 may house a spring or additional delivery mechanism configured with the subdermal needle 412 so as to provide additional force for penetrating a subdermal region. Additionally, the loading mechanism may further protect and sheath the subdermal needle from accidental sticks.

In other aspects, the subdermal needle 412 and the lead wire are mated with a solder alloy. In other aspects they may be joined by a metallic paste, or integrated through wrapping, or in any manner in which electrical signals can travel from the needle portion through to the lead wire, and ultimately to a monitor for displaying the received signals.

In additional aspects, the subdermal needle electrode may further comprise an insulation wrap, wherein the insulation wrap is wrapped around the solder alloy, or the lead wire itself, and including a portion of the subdermal needle. Such a wrapping may be due to insulating the electrical communication pathway, or for protection, or for enhancing the fit within the inner portion of the housing. In other aspects, the outer portion of the housing encases the subdermal needle to prevent needle sticks when not compressed, while driving the needle downwards when it is compressed. Thus the outer portion is semi-rigid, but capable of being compressed and maintaining integrity to return to its original position.

Continuing, in further aspects the subdermal needle electrode may be removed from the patient by retracting the subdermal needle. In this aspect, the steps comprise removing the subdermal needle electrode by moving in an opposite direction from insertion, wherein the removing of the subdermal needle allows the subdermal needle to retract back into the outer portion 406 of the subdermal needle electrode housing 400, guarding the subdermal needle 412 from improper needle sticks.

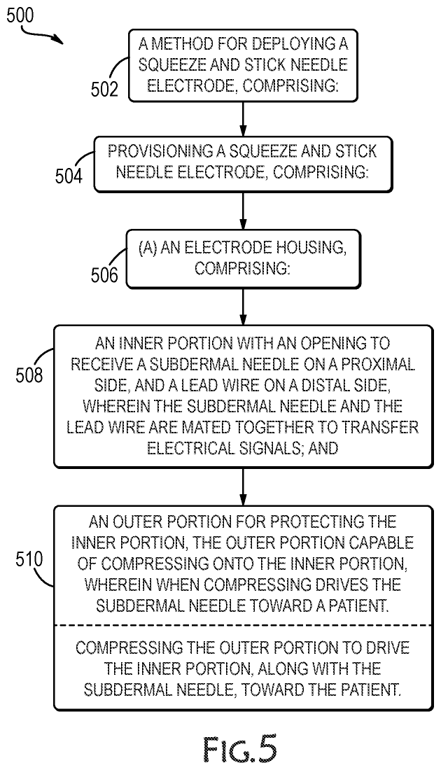

Referring now to FIG. 5, in other aspects, a method for deploying a squeeze and stick needle electrode 502, is disclosed. The method 500 begins by provisioning a squeeze and stick needle electrode 504, comprising: (a) an electrode housing, the electrode housing comprising: (i) an inner portion with an opening to receive a subdermal needle on a proximal side, and a lead wire on a distal side, wherein the subdermal needle and the lead wire are mated together to transfer electrical signals 506; and (ii) an outer portion for protecting the inner portion, the outer portion capable of compressing onto the inner portion, wherein when compressing drives the subdermal needle toward a patient 508. Next, compressing the outer portion to drive the inner portion, along with the subdermal needle, toward the patient 510.

In additional aspects, the method further comprises releasing the compressing forces once the subdermal needle has entered the patient. By releasing the compressing forces, the outer housing returns to its original position or non-compressed state. Further, there may be nodules or a latching mechanism that may hold the outer portion in a compressed state, until compressed further to release to a non-compressed state.

In additional aspects, the method may further comprise removing the subdermal needle by moving the needle in an opposite direction from insertion, wherein the removing of the subdermal needle allows the subdermal needle to move back into the outer portion guarding the subdermal needle from improper needle sticks. Thus, the removal operation does not require additional steps such as further compression of the outer housing. In additional aspects, with a locking mechanism, the removal may require additional compression of the outer housing so as to release the outer housing and allow the inner housing to return to its pre-deployment or original state.

It should be emphasized that the above-described embodiments of the present disclosure are merely possible examples of implementations set forth for a clear understanding of the principles of the disclosure. Many variations and modifications may be made to the above-described embodiments without departing substantially from the scope and principles of the disclosure. All such modifications and variations are intended to be included herein within the scope of this disclosure and protected by the following claims.

Therefore, the following is claimed:

1. A squeeze and stick subdermal needle electrode, comprising:
(a) an electrode housing, comprising:
(i) an inner portion comprising a semi-rigid projection with a top surface sloped downward from a medial line, the semi-rigid projection with an opening to receive a subdermal needle on a proximal side, and a lead wire on a distal side, wherein the subdermal needle and the lead wire are mated together to transfer electrical signals; and
(ii) an outer portion comprising a left side and a right side of semi-rigid material for protecting the inner portion, the outer portion capable of compressing into the inner portion, wherein the compressing drives the left side and the right side of the outer portion into the top surface of the semi-rigid projection which drives the subdermal needle toward a patient.

2. The subdermal needle electrode of claim 1, wherein the electrode housing is comprised of a polymeric material.

3. The subdermal needle electrode of claim 1, wherein the inner portion is comprised of a polymeric material and the outer portion is comprised of a metal.

4. The subdermal needle electrode of claim 1, wherein the subdermal needle is comprised of stainless steel and a non-tip portion of the subdermal needle has a protective coating.

5. The subdermal needle electrode of claim 1, further comprising the outer portion having an angular left side and right side that when acted upon compresses the outer portion into the inner portion at a respective angle that causes the outer portion to slide above the inner portion to drive the subdermal needle toward the patient.

6. The subdermal needle electrode of claim 1, further comprising the outer portion having the left side and the right side, wherein when the left side and the right side are connected to form a circle that provides protection from the subdermal needle.

7. The subdermal needle electrode of claim 1, wherein the proximal side of the inner portion is configured with an adhesive to secure and support the subdermal needle.

8. The subdermal needle electrode of claim 1, wherein the subdermal needle and the lead wire are mated with a solder alloy.

9. The subdermal needle electrode of claim 8, further comprising an insulation wrap, wherein the insulation wrap is wrapped around the solder alloy, the subdermal needle, and the lead wire.

10. The subdermal needle electrode of claim 1, wherein the outer portion is open on one end to allow movement of the subdermal needle to enter into subdermal tissue of the patient.

11. A method for deploying a squeeze and stick needle electrode, comprising:
provisioning a squeeze and stick subdermal needle electrode, comprising:
(a) an electrode housing, comprising:
(i) an inner portion comprising a semi-rigid projection with a top surface sloped downward from a medial line, the semi-rigid projection with an opening to receive a subdermal needle on a proximal side, and a lead wire on a distal side, wherein the subdermal needle and the lead wire are mated together to transfer electrical signals; and
(ii) an outer portion comprising a left side and a right side of semi-rigid material for protecting the inner portion, the outer portion capable of compressing into the inner portion;
compressing the left side and the right side of the outer portion into the top surface of the semi-rigid projection, which drives the subdermal needle toward a patient.

12. The method of claim 11, further comprising releasing compressing forces from the left side and the right side of the outer portion once the subdermal needle has entered the patient.

13. The method of claim 11, further comprising removing the subdermal needle electrode by moving in an opposite direction from insertion, wherein the removing of the subdermal needle allows the subdermal needle to retract back into the outer portion guarding the subdermal needle from improper needle sticks.

14. The method of claim 11, wherein the electrode housing is comprised of polymeric material.

15. The method of claim 11, wherein the inner portion is comprised of a polymeric material and the outer portion is comprised of a metal.

16. The method of claim 11, wherein the subdermal needle is comprised of stainless steel and a non-tip portion of the subdermal needle has a protective coating.

17. The method of claim 11, further comprising the outer portion with an angular left side and right side that when acted upon compresses the outer portion into the inner portion at a respective angle that causes the outer portion to slide above the inner portion to drive the subdermal needle toward the patient.

18. The method of claim 11, further comprising the outer portion having the left side and the right side, wherein when the left side and the right side are connected to form a circle that provides protection from the subdermal needle.

19. The method of claim 11, wherein the outer portion is open on one end to allow movement for the subdermal needle to enter into subdermal tissue of the patient.
